(19) United States
(12) Plant Patent Application Publication
(10) Pub. No.: US 2006/0117441 P1
(43) Pub. Date: Jun. 1, 2006

(54) GERANIUM PLANT NAMED 'GRAFIRE'

(50) PELARGONIUM ZONALE; GERANIUM

(75) Inventor: Angelika Utecht, Montabaur (DE)

Correspondence Address: FOLEY AND LARDNER LLP;SUITE 500, 3000 K STREET NW, WASHINGTON, DC, 20007, US

(73) Assignee: Florfis AG

(21) Appl. No.: 10/998,664

(22) Filed: Nov. 30, 2004

(51) Int. Cl. A01H 5/00

(52) U.S. Cl. PLT/330

(57) ABSTRACT

A new and distinct cultivar of geranium plant named ‘Grafire’ particularly characterized by the combined features of brilliant red, star-shaped, single-type flowers; numerous, umbrella-shaped inflorescences, well above the foliage; medium sized, green, fan-shaped leaves with strong zonation; medium sized, rounded and well-branched plant habit; an beginning of flowering in mid season with continuous flowering.

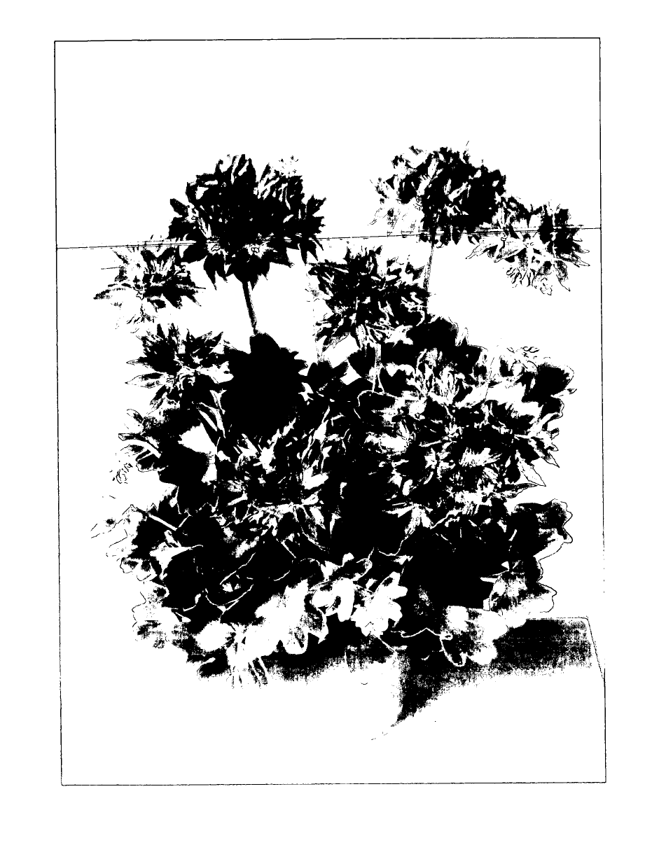

### Genus and Species of the Plant Claimed

Pelargonium zonale L'Heritier hybrid)

### Variety Denomination

‘Grafire’

### BACKGROUND OF THE INVENTION

The present invention comprises a new and distinct cultivar of geranium, botanically known as Pelargonium zonale, and hereinafter referred to by the cultivar name ‘Grafire’.

‘Grafire’ is a product of a planned breeding program which had the objective of creating new stellar-type geranium cultivars in various flower colors, in combination with early flowering, zoned foliage, and about medium sized, rounded and well-branched plant habit.

‘Grafire’ originated from a hybridization made by the inventor, Angelika Utecht, in a controlled breeding program in Hillscheid, Germany, in 1999. The female parent was an unpatented hybrid seedling, no. 88-380-1, having small, red single-type flowers, medium green foliage with distinct zonation, and relatively compact plant habit. The male parent of ‘Grafire’ was the unpatented commercial variety ‘Vectis Glitter’, with single-type, white flowers with irregularly spread small red dots or fine stripes, large inflorescences, and vigorous growth habit.

‘Grafire’ was selected as one flowering plant within the progeny of the stated cross by Angelika Utecht in 2000 in a controlled environment in Fuerteventura, Canary Islands, Spain.

The first act of asexual reproduction of ‘Grafire’ was accomplished when vegetative cuttings were taken from the initial selection in the fall of 2000 in a controlled environment in Fuerteventura, Spain, by, or under the supervision of, Angelika Utecht.

Horticultural examination of plants grown from cuttings of the plant initiated in May of 2001, in Hillscheid, Germany, and continuing thereafter, has demonstrated that the combination of characteristics as herein disclosed for ‘Grafire’ are firmly fixed and are retained through successive generations of asexual reproduction.

### BRIEF SUMMARY OF THE INVENTION

‘Grafire’ has not been observed under all possible environmental conditions. The phenotype may vary significantly with variations in environment such as temperature, light intensity and day length. The following observations, measurements, and comparisons describe plants grown in Hillscheid, Germany, under greenhouse conditions which approximate those generally used in commercial practice.

The following traits have been repeatedly observed and are determined to be basic characteristics of ‘Grafire’ in combination distinguish this geranium as a new and distinct cultivar:

1. brilliant orange-red, single-type flowers;

2. medium sized, umbrella-shaped inflorescences, well above the foliage;

3. medium sized, fan-shaped leaves with strong zonation;

4. medium sized and well-branched plant habit; and

5. beginning of flowering in mid season.

Of the many commercial cultivars known to the present inventor, the most similar in comparison to ‘Grafire’ are the patented variety ‘Fiwoscarl’ (U.S. Plant Pat. No. 13,303) and the unpatented variety ‘Red Flash’, also known by breeder's ref. KLEP01002.

In comparison to ‘Fiwoscarl’, ‘Grafire’ has similar flower size and color, but roughly semi-circular leaf blades whereas the leaves of ‘Fireworks Scarlet’ are kidney-shaped. Further, ‘Fireworks Scarlet's plant habit is generally taller and with a higher foliage canopy than ‘Grafire’.

In comparison to ‘Red Flash’, with numerous small inflorescence, and compact plant habit, ‘Grafire’ has larger and flatter shaped umbels, foliage with less strong zonation, and taller, medium sized, plant habit.

### BRIEF DESCRIPTION OF THE DRAWING

The accompanying photographic drawing shows typical flower and foliage characteristics of ‘Grafire’ with colors being as true as possible with an illustration of this type. The photographic drawing shows a side view perspective of a flowering potted plant of ‘Grafire’ with leaves, buds and inflorescences.

### DETAILED BOTANICAL DESCRIPTION

The measurements were taken in Hillscheid, Germany, in mid May, 11 weeks after planting of rooted cuttings. The plants were growing in 14 cm pots, they had not been pinched.

In the following description, color references are made to The Royal Horticultural Society Color Chart. The color values were determined indoors from plants growing in a green-house in May 2003 in Hillscheid, Germany.

### Parentage:

Male parent: ‘Vectis Glitter, unpatented commercial variety.
Female parent: Unpatented, hybrid seedling no. 88-380-1.

### Classification:

Pelargonium zonale L'Heritier (hybrid).

### Propagation:

Vegetative cuttings.

### Inflorescense

Umbel:
   Shape.—Umbrella-shape.
   Average diameter.—83 mm.
   Average depth.—45 mm.
   Peduncle length.—159 mm.
   Peduncle color.—Light grey-red, RHS 179 B, lower part yellow-green, RHS 144 B.
   Pedicel.—25 mm in length, usually with a spur.
   Pedicel color.—Most parts grey-red infused, RHS 180 B, near the base yellow-green, RHS 152 D.
   Number of flowers per umbel.—20-25.
Corolla:
   Average diameter.—40 mm.
   Form.—Single-type.
   Shape.—Star-shape, with the 2 upper petals narrower than the lower petals.
   Number of petals.—5.
   Number of petaloids.—None.
   Shape of petals.—Roughly elliptical, with acute base and acuminate tips, with weak lobes at the side margins.
   Size of petals.—Upper petals: 22 mm long, 8 mm wide; lower petals: 20 mm long, 10 mm wide.
   Color (general tonality from a distance of three meters).—Red.
   Color of upper petals.—Red, RHS 44 A.
   Markings of upper petals.—No distinct markings.
   Color of lower petals.—Red, RHS 44 A.
   Markings of lower petals.—None.
   Color of lower surface of petals.—Red, RHS 41 A.
   Color of sepals Outer surface.—yellow-green, RHS 144 B; inner surface: yellow-green, RHS 144 B.
   Number of sepals.—5.
   Shape of sepals.—Ensiform, acute tip, sessile base, surface with very short pubescence, margin entire.
   Size of sepals.—9-10 mm long, 3 mm wide for the largest upper sepal, 2 mm in width for the other sepals.
Bud (just prior to petals unfolding):
   Shape.—Narrow, elliptical.
   Color of sepals.—Yellow-green, RHS 144 B.
   Color of petals.—Red, RHS 45 B.
   Length.—13-14 mm.
   Width.—6 mm.

### Reproductive Organs

Androecium: 5 fertile anthers, moderate pollen, orange-red, RHS 30 A, filaments white, RHS 155 D, to light-pink, RHS 52 D.
Gynoecium: One pistil, whitish style, RHS 155 D to red-purple, RHS 65 D, stigma 5- 6-lobed and pink, RHS 50 A, in color.
Fertility/seed set: No seed set observed.
Spring flowering response period: In Hillscheid, Germany, in 2003 plants had on average 0.8 flowers opened 9 weeks after planting of rooted cuttings.
Outdoor flower production: Continuously and rich flowering, the flower count in 2003 in Hillscheid, Germany, indicated about 20 inflorescences per plant in late summer.
Durability Good stability of flower color, no fading, fair rain resistance.
Lastingness of the individual flower: About 7-8 days at 18° C., about 16 days for the umbel.
Fragrance: None.

### Plant

Foliage:
   Shape.—Roughly semi-circular, flabellate, with distinct lobes, base wide open, forming an angle of more than 180°.
   Margin.—Serrate, respective with incisions.
   Texture.—Upper surface smooth, dull.
   Size of leaf.—87 mm wide, 50 mm long.
   Color of upper surface.—Green, approximately RHS 143 B.
   Color of zonation.—Strong, brown (greyed-orange), about RHS 166 A.
   Color of lower surface.—Green, RHS 143 B to 143 C.
   Petioles.—65-75 mm long, 2.0-2.5 mm diameter, light green in color, approximately RHS 143 B.
General appearance and form:
   Stem color.—Mainly light green, RHS 143 C; in places slightly grayish-red infused, RHS 179 B.
   Internode length.—15-25 mm.
   Branching pattern.—12 branches.
   Size of plants.—15.3 cm, 27.3 cm wide (11-week-old plants, as described, measured from the top of the soil (base of the main stem) to the surface of the foliage canopy, without inflorescences).

I claim:

1. A new and distinct cultivar of geranium plant named ‘Grafire’, as described and illustrated herein.
